(19) United States
(12) Patent Application Publication
(10) Pub. No.: US 2005/0100593 A1
(43) Pub. Date: May 12, 2005

(54) Capsule film composition and gelatin capsule

(75) Inventor: Kiyonori Furuta, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: AJINOMOTO CO., INC.

(21) Appl. No.: 10/983,803

(22) Filed: Nov. 9, 2004

(30) Foreign Application Priority Data
Nov. 10, 2003 (JP) 2003-380092

(51) Int. Cl. A61K 9/64

(52) U.S. Cl. 424/456

(57) ABSTRACT

A gelatin capsule, formed of a film composition containing gelatin and polyglutamic acid, encompassing a mineral ingredient as a part of the contents of the capsule, together resulting in easy absorption of the mineral ingredient upon administration of the capsule. The composition of the film comprising the gelatin capsule inhibits an insolubilization phenomenon in the film that occurs during storage of the gelatin capsule, and composition of the film allows production of the capsule by an existing production line. Further, the gelatin capsule can easily be administered orally, and minerals contained in the capsule, such as calcium and iron, can effectively be absorbed in the living body.

### BACKGROUND OF THE INVENTION

1. Field of the Invention

The present invention relates to a gelatin capsule which is widely used in the field of pharmaceutical preparations, foods, health foods and the like. More specifically, it relates to a capsule film composition in which an insolubilization phenomenon that is observed in capsule films after long-term storage of a gelatin capsule is prevented, and a gelatin capsule formed of this film composition.

2. Description of the Related Art

Capsules using gelatin as a film material, namely gelatin capsules have been often used not only in the field of pharmaceutical preparations, but also in the field of health foods such as nutrition supplemental foods and functional foods because of the increasing interest in health in recent years. Gelatin capsules have been used for protecting and stabilizing ingredients contained in pharmaceutical preparations and foods, masking a taste or a smell and the like.

However, in the capsules using gelatin as a film material, a known problem is that crosslinking occurs in the gelatin contained in the film during the long-term storage, thereby causing an insolubilization phenomenon of the film. In order to solve such a problem, it has been known, for example, to use gelatin obtained by chemically modifying an amino group in a gelatin molecule with an organic acid as a film substrate (refer to, for example, JP-A-2000-44465). This document describes that capsules using such a chemically modified gelatin are problematic in that they tend to be sticky and are hard to handle, and a polyglucose compound such as starch has to be added to prevent these problems. However, so far as disintegration data after storage at 40° C. for 2 months is seen, the use of this method has been still unsatisfactory in preventing the insolubilization of the film.

As other edible capsules, capsules containing polyglutamic acid have been known. As film capsules made only of polyglutamic acid, for example, edible capsules using as a film a polyglutamic acid crosslinked product, obtained by crosslinking polyglutamic acid with a metal such as alum, have been known (refer to a pamphlet of WO 03/049771). However, when a film sheet is formed from a solution containing the polyglutamic acid crosslinked product in the production of edible capsules using the polyglutamic acid crosslinked product as a film, change to sol which is easily processed by heating as in gelatin does not take place. Accordingly, a drying step (for example, at room temperature and 45% RH for 24 hours) is sometimes required as an extra step, in comparison to an ordinary method for forming a film sheet for gelatin capsules. Consequently, in the production of the film capsules made only of polyglutamic acid, such problems in equipment and efficiency have remained unresolved that an existing production line of gelatin capsules cannot be diverted directly and the addition of another step might considerably prolong the production time.

Meanwhile, Japanese people are likely to be short of calcium, iron, zinc, copper, magnesium and the like among mineral ingredients constituting the living body. Especially, calcium is an essential nutrient required at a growth age, and also indispensable for adults because shortage of calcium leads to osteoporosis. Under these circumstances, for the purpose of satisfying a nutritional requirement of calcium, a large number of foods and drinks enriched with an inorganic calcium salt or a calcium powder, calcium preparations (powders or tablets) and the like have been commercially available.

Regarding supplementation of calcium, the study and the development of auxiliaries for accelerating absorption of calcium, besides calcium per se, have been performed. Especially, as a material showing an effect of accelerating absorption of calcium by increasing a concentration of soluble calcium in the small intestine, poly-γ-glutamic acid, poly-α-glutamic acid and the like are mentioned. Poly-γ-glutamic acid has been known as a substance present in a viscous substance of natto and as an ingredient which microorganisms belonging to the genus Bacillus, such as Bacillus natto, secrete extracellularly. These polyglutamic acids have an effect of solubilizing minerals in the lower portion of the small intestine, making it possible to accelerate intestinal absorption of minerals (refer to, for example, JP-A-59-162843 and Japanese Patent No. 3232718).

As stated above, there are several types of calcium preparations, which have merits and demerits, though. Further, since the calcium preparations containing calcium as an active ingredient take a dosage form of powders or capsules, a combination with other ingredients has been limited to a combination with solid materials. Thus, calcium preparations which have a dosage form capable of combining with liquid materials and are easy of oral administration have been in demand.

### SUMMARY OF THE INVENTION

The invention aims to provide, for solving the foregoing problems, a capsule film composition and a capsule made therefrom which can inhibit an insolubilization phenomenon of a film from occurring during storage of a gelatin capsule. It also aims to find a composition which can be produced with an existing production line without the need of providing a drying step for forming a film sheet as in the production of the ordinary polyglutamic acid film capsules. Moreover, it aims to provide a gelatin capsule which is easily administered orally and that allows minerals taken in, such as calcium and iron, to be effectively absorbed in the living body.

Under these circumstances, the present inventors have assiduously conducted investigations, and have consequently found that an insolubilization phenomenon which might occur after long-term storage can greatly be inhibited by incorporating polyglutamic acid as a film component of a gelatin capsule, that a film containing polyglutamic acid can be produced in an existing gelatin capsule production line, and further, that capsule preparations that accelerate absorption of minerals such as calcium can be formed. That is, the present inventors have found that a combination of gelatin and polyglutamic acid as film components of the capsule can solve the foregoing three problems at the same time. These findings have led to the completion of the invention.

That is, the invention provides the following capsule film composition and gelatin capsule.

(1) A capsule film composition comprising gelatin and polyglutamic acid.

(2) A gelatin capsule comprising the capsule film composition recited in (1).

(3) The gelatin capsule recited in (2), wherein the polyglutamic acid is poly-γ-glutamic acid and/or poly-α-glutamic acid.

(4) The gelatin capsule recited in (2) or (3), which includes a capsule content ingredient that includes a mineral ingredient.

(5) The gelatin capsule recited in (4), wherein the mineral ingredient is calcium and/or iron.

The invention provides a capsule film composition comprising gelatin and polyglutamic acid, and a gelatin capsule formed from the capsule film composition, whereby the insolubilization phenomenon of the capsule film that occurs during storage of some types of gelatin capsules can be inhibited and the production is enabled by diverting an existing gelatin production line. Further, when polyglutamic acid is incorporated in the capsule film, other active ingredients can be contained in larger amounts in comparison to the case where polyglutamic acid is a content ingredient. Still further, not only a combination with a solid material but also a combination with a liquid material, a material having a different taste or smell or a material easily oxidizable with oxygen is possible, and preparations with a high degree of freedom in designing a recipe of active ingredients is provided. Especially when mineral ingredients such as calcium and iron are incorporated in contents included in capsules, they can easily be administered orally, and the mineral ingredients taken in are effectively absorbed in the living body by being solubilized in the intestinal tract with polyglutamic acid. Thus, effects of supplementing minerals in children at a growth age, preventing osteoporosis at an old age and the like can be expected.

### DESCRIPTION OF THE PREFERRED EMBODIMENTS

The preferred embodiments of the invention are described below. (1) A capsule film composition comprising gelatin and polyglutamic acid, (2) a gelatin capsule formed of this film composition, and (3) a gelatin capsule excellent in easy absorption of minerals and comprising the capsule as a film and a capsule content ingredient included in the capsule, constituents of the capsule film being gelatin and polyglutamic acid, and the capsule content ingredient containing a mineral ingredient.

The gelatin used as a main component of the capsule film in the invention is not particularly limited. For example, gelatin which allows a sol-gel change according to thermal change and is obtained using bovine, swine, poultry, fish or the like as a raw material, can be used. Usually, gelatin having optimum jelly strength can selectively be used according to the use. In the gelatin capsules of the invention, optimum jelly strength can be selected according to the type of capsules. For example, in case of a soft capsule, the gel strength is from 100 to 250 bloom, preferably from 120 to 200 bloom. In case of a seamless capsule, it is from 150 to 350 bloom, preferably from 200 to 300 bloom.

Polyglutamic acid used as the capsule film component of the invention is not particularly limited. Examples thereof include synthetic poly-α-glutamic acid, poly-γ-glutamic acid which is present in a viscous material of natto or which microorganisms belonging to the genus Bacillus, such as Bacillus natto, extracellularly secrete, salts thereof and the like. These polyglutamic acids are effective for solubilizing minerals in the lower portion of the small intestine, and accelerate intestinal absorption of minerals. When they are used as food materials, poly-α-glutamic acid might be decomposed with a protease in migration within the intestinal tract, which depends on conditions such as an amount, though. Accordingly, from the standpoint of providing a high effect of accelerating intestinal absorption of minerals, poly-γ-glutamic acid which is present in a viscous material of natto or which microorganisms belonging to the genus Bacillus, such as Bacillus natto, extracellularly secrete is preferable.

The molecular weight of polyglutamic acid is not particularly limited. It is preferably from 3,000 to 1,000,000. When it exceeds 1,000,000, a step such as dissolution becomes difficult. When it is less than 3,000, no effect of accelerating absorption of minerals is obtained. The molecular weight employed in the invention is a weight average molecular weight measured by a gel filtration-light scattering method (GPC-MALLS method: Dawn DPS manufactured by Wyatt Technology). Polyglutamic acid is generally in the form of a sodium salt. Polyglutamic acid used in the invention may be in the form of the sodium salt or other salts, or may be carboxyl group-free polyglutamic acid.

The gelatin capsule of the invention may take any form of various capsules such as a hard capsule, a soft capsule, a seamless soft capsule. Further, the polyglutamic acid-containing gelatin capsule of the invention can be produced by an ordinary method according to the use, purpose, and the required shape and size. That is, any form of various capsules such as a hard capsule, a soft capsule and a seamless soft capsule is available, and they are produced respectively by prescribed methods.

For example, as a method for producing a hard capsule, a dipping method in which a hard capsule is produced by dipping a mold (mold pin) in a film solution and drying the same can be used.

As a method for producing a soft capsule, a rotary die method which is a type of a stamping method can be used. That is, it is a method in which capsule formation, filling of contents and heat sealing are conducted simultaneously using two sheets.

As a method for producing a seamless soft capsule, a method in which curing is conducted in liquid, a type of a dripping method, can be used. That is, it is a method in which a capsule content solution flows from an inside nozzle of a double or more multiple nozzle and a capsule film solution from an outside nozzle at a fixed flow rate and this two-layer liquid stream is cut at fixed intervals to give droplets, after which an outside film layer is gelled to form a capsule.

As a method for producing microcapsules, a coacervation (phase separation) method, an emulsification (stirring emulsification or ultrasonic emulsification) method, a spray-drying method and the like can be used.

The form, the size and the like of the capsule are not particularly limited. With respect to the form, a capsule having a round form, an oval form, an oblong form, a tubular form or a tear drop form can be produced. With respect to the size, a capsule having a size of from several micrometers to several centimeters can be produced. The gelatin capsule can be produced by an ordinary method according to the use, purpose, and the required form and size.

In the invention, the optimum contents of gelatin and polyglutamic acid occupied in the constituents of the film vary with the type of the capsule and the jelly strength of gelatin used. They can be determined during the production of the capsule unless the film properties of the capsule are impaired. The total amount of gelatin and polyglutamic acid is preferably in the range of from 60 to 100% of the overall amount of the film. When it is less than 60%, the film strength is not maintained in production of the capsule, which makes the production difficult. It is preferably from 60 to 85%, more preferably from 65 to 80%. The optimum content of polyglutamic acid in the constituents of the film varies with the type of the capsule and the jelly strength of gelatin used. It can be determined unless the film properties are impaired. The mixing ratio of gelatin to polyglutamic acid (as a weight ratio) is usually in the range of from 99:1 to 60:40. A preferable range can be determined according to methods for production of various capsules and film properties required. For example, in case of a soft capsule using a gelatin film, the mixing ratio of gelatin to polyglutamic acid (as a weight ratio) is preferably in the range of from 99:1 to 60:40. When the ratio of gelatin is higher than 99:1, it is difficult to secure ability of inhibiting insolubilization with polyglutamic acid. Meanwhile, when the ratio of polyglutamic acid is higher than 60:40, the gel strength is insufficient, which makes the production of the capsule difficult. In view of securing the ability of inhibiting insolubilization and the production of the capsule, the mixing ratio of gelatin to polyglutamic acid is preferably from 95:5 to 65:35, more preferably from 90:10 to 65:35.

In case of the seamless capsule, the mixing ratio of gelatin to polyglutamic acid (as a weight ratio) is preferably from 99:1 to 70:30. When the ratio of gelatin is higher than 99:1, it is difficult to secure the ability of inhibiting insolubilization with polyglutamic acid. Meanwhile, when the ratio of polyglutamic acid is higher than 70:30, the gel strength is insufficient, which makes the production of the capsule difficult. In view of securing the ability of inhibiting insolubilization and the production of the capsule, it is more preferably from 95:5 to 75:25.

In addition to the gelatin and polyglutamic acid components of the invention, other components which are ordinarily used as film components can be incorporated unless the effects of the invention are impaired. Known additives, for example, a plasticizer such as glycerin, a colorant and a screening agent are available.

The content ingredient inside of the capsule can be incorporated in the gelatin capsule of the invention either separately from, or simultaneously with, the production of the capsule. As the content ingredient, other ingredients which are used as content ingredients of capsule preparations, whether solid or liquid, can be incorporated unless the effects of the invention are impaired. Examples thereof include known nutrient-related materials such as proteins, vitamins and lipids, herbs, functional food materials such as physiologically active peptides, and the like.

Mineral ingredients used as content ingredients of the gelatin capsule in the invention include a part or all of minerals indispensable for the living body, such as calcium, iron, magnesium, zinc and copper. The form of minerals used is not particularly limited, and it may be a liquid form or a solid form. Examples of calcium include synthetic food additives such as calcium chloride, calcium carbonate, calcium hydroxide, calcium sulfate, tricalcium phosphate, calcium hydrogenphosphate, calcium dihydrogenphosphate, calcium citrate, calcium gluconate, calcium lactate, calcium pantothenate and calcium dihydrogenpyrophosphate, natural calciums such as shell calcium and bone calcium. Examples of iron include synthetic food additives such as ferric chloride, iron citrate, ferrous citrate, iron aluminum citrate, iron gluconate, iron lactate, ferrous pyrophosphate, ferric pyrophosphate and ferrous sulfate, and natural irons such as hem iron. Magnesium and other minerals may be chloride compounds thereof, mineral powders or minerals incorporated into yeast.

The weight ratio of mineral ingredients and polyglutamic acid is not particularly limited, and it can optionally be determined on the basis of a nutritional requirement per day, an average intake and the like. For example, regarding calcium, a nutritional requirement per day for an adult is 700 mg, and an average intake for a people is 547 mg (2000th National Nutrition Research). Accordingly, calcium is calculated to be deficient by approximately 150 mg. When 200 mg of calcium is taken in from a polyglutamic acid-containing gelatin capsule for covering the deficiency, polyglutamic acid required can be used in the range of from 10 mg to 500 mg. When the amount of polyglutamic acid is smaller than 10 mg, no sufficient effect for accelerating absorption of calcium is obtained. When it is larger than 500 mg, a sufficient effect is already obtained, and no higher effect can be expected. The mixing amount of polyglutamic acid as a film constituent is preferably from 20 mg to 300 mg, more preferably from 30 mg to 200 mg.

As other capsule content ingredients included in the mineral-containing gelatin capsule of the invention, other ingredients used as contents of capsule preparations can be incorporated unless the effects of the invention are impaired. For example, known nutrition-related materials such as proteins, vitamins and lipids, herbs and functional food materials such as physiologically active peptides are available. Specifically, in case of calcium, vitamin K which is deemed to promote osteogenesis by binding calcium in blood to bones, isoflavon having activity of inhibiting bone absorption by which calcium stored in bones is released to blood can be used in combination. Consequently, it can be expected that calcium taken in orally is utilized more efficiently and generally strengthens bones to allow prevention of osteoporosis or the like.

### EXAMPLES

The invention is illustrated more specifically below by referring to EXAMPLES. However, the invention is not limited to the following EXAMPLES.

In EXAMPLES 1, 2 and 3 and COMPARATIVE EXAMPLE 1, film solutions and content solutions were prepared as mentioned below, and formed into soft capsules with a continuous rotary die-type capsule filling machine.

### Example 1

Starting materials shown in the following film solution formulation 1 were heat-dissolved at 65° C., and the solution was degassed to prepare a film solution such that viscosity at 60° C. became 20,000 cps. Further, starting materials shown in the following content solution formulation 1 were mixed with stirring, and then uniformly dispersed with a homogenizer to prepare a content solution.

<Film solution formulation 1>

| | parts by weight |
|---|---|
| gelatin (jelly strength = 150 bloom) | 85 |
| poly-γ-glutamic acid (molecular weight 30,000) | 15 |
| glycerin | 35 |
| purified water | 80 |

<Content solution formulation 1>

| | parts by weight |
|---|---|
| shell calcium | 100 |
| soybean oil | 200 |

Using the resulting film solution and content solution, capsule formation was performed with a continuous rotary dye-type capsule filling machine using a mold of OVAL No. 5. At this time, the time that lapsed from the formation of the film sheet to the capsule formation was approximately 4 minutes. Then, the capsules were dried to produce soft capsules. Each of the resulting soft capsules had a long diameter of 12.8 mm, a short diameter of 8.1 mm, a total weight of 463 mg, a content weight of 303 mg, a film thickness of 0.6 mm and a film water content of 10.1%.

### Example 2

Starting materials shown in the following film solution formulation 2 were heat-dissolved at 65° C., and the solution was degassed to prepare a film solution such that viscosity at 60° C. became 20,000 cps. Further, starting materials shown in the following content solution formulation 2 were mixed with stirring, and then uniformly dispersed with a homogenizer to prepare a content solution.

<Film solution formulation 2>

| | parts by weight |
|---|---|
| gelatin (jelly strength = 150 bloom) | 70 |
| poly-γ-glutamic acid (molecular weight 30,000) | 30 |
| glycerin | 35 |
| purified water | 80 |

<Content solution formulation 2>

| | parts by weight |
|---|---|
| shell calcium (Ca content 38%) | 100 |
| soybean oil | 200 |

Using the resulting film solution and content solution, capsule formation was performed with a continuous rotary dye-type capsule filling machine using a mold of OVAL No. 5. At this time, the time that lapsed from the formation of the film sheet to the capsule formation was approximately 4 minutes. Then, the capsules were dried to produce soft capsules. Each of the resulting capsules had a long diameter of 12.7 mm, a short diameter of 8.2 mm, a total weight of 483 mg, a content weight of 302 mg, a film thickness of 0.6 mm and a film water content of 7.6%.

### Example 3

Starting materials shown in the following film solution formulation 3 were heat-dissolved at 65° C., and the solution was degassed to prepare a film solution such that viscosity at 60° C. became 20,000 cps. Further, starting materials shown in the following content solution formulation 3 were mixed with stirring, and then uniformly dispersed with a homogenizer to prepare a content solution.

<Film solution formulation 3>

| | parts by weight |
|---|---|
| gelatin (jelly strength = 150 bloom) | 85 |
| poly-γ-glutamic acid (molecular weight 30,000) | 15 |
| glycerin | 30 |
| purified water | 80 |

<Content solution formulation 3>

| | parts by weight |
|---|---|
| shell calcium (Ca content 38%) | 80 |
| soybean isoflavon (effective content 40%) | 20 |
| vitamin K2 (effective content 0.15%) | 5 |
| soybean oil | 195 |

Using the resulting film solution and content solution, capsule formation was performed with a continuous rotary dye-type capsule filling machine using a mold of OVAL No. 5. At this time, the time that lapsed from the formation of the film sheet to the capsule formation was approximately 4 minutes. Then, the capsules were dried to produce soft capsules. Each of the resulting soft capsules had a long diameter of 11.8 mm, a short diameter of 8.4 mm, a total weight of 449 mg, a content weight of 301 mg, a film thickness of 0.51 mm and a film water content of 9.1%.

### Comparative Example 1

Starting materials shown in the following film solution formulation 4 were heat-dissolved at 65° C., and the solution was degassed to prepare a film solution such that viscosity at 60° C. became 20,000 cps. Further, starting materials shown in the following content solution formulation 4 were mixed with stirring, and then uniformly dispersed with a homogenizer to prepare a content solution.

<Film solution formulation 4>

| | parts by weight |
|---|---|
| gelatin (jelly strength = 150 bloom) | 100 |
| glycerin | 35 |
| purified water | 80 |

<Content solution formulation 4>

| | parts by weight |
|---|---|
| shell calcium (Ca content 38%) | 100 |
| soybean oil | 200 |

Using the resulting film solution and content solution, capsule formation was performed with a continuous rotary dye-type capsule filling machine using a mold of OVAL No. 5. At this time, the time that lapsed from the formation of the film sheet to the capsule formation was approximately 4 minutes. Then, the capsules were dried to produce soft capsules. Each of the resulting soft capsules had a long diameter of 12.6 mm, a short diameter of 8.1 mm, a total weight of 451 mg, a content weight of 301 mg, a film thickness of 0.59 mm and a film water content of 10.2%.

The results of evaluating the soft capsules obtained by the foregoing method are shown in Test EXAMPLES 1 to 3.

### Test Example 1

### Disintegration Test

The capsules obtained in EXAMPLE 1 and COMPARATIVE EXAMPLE 1 were put in a sealed glass container, and stored in an atmosphere of 40° C. and 75% RH for 3 months and 6 months. The samples before storage (initial value) and after 3 months and 6 months were tested according to a disintegration test method prescribed in the manual of Japanese Pharmacopoeia using a disintegration tester (NT-4HSF manufactured by Toyama Sangyo). Regarding 9 capsules, the disintegration time was measured under respective conditions, and the resulting average values are shown in TABLE 1.

TABLE 1

| | | EXAMPLE 1 | | COMPARATIVE EXAMPLE 1 | |
|---|---|---|---|---|---|
| | | PGA-containing film capsule | | Gelatin capsule | |
| | | Test solution | | | |
| | | Artificial gastric | Artificial | Artificial gastric | Artificial |
| | | juice | intestinal juice | juice | intestinal juice |
| Disintegration | Initial | 7 minutes and | 8 minutes and | 7 minutes and | 9 minutes and |
| time | value | 28 seconds | 41 seconds | 32 seconds | 10 seconds |
| | After 3 | 11 minutes and | 15 minutes and | 11 minutes and | 20 minutes and |
| | months | 43 seconds | 13 seconds | 17 seconds | 1 second |
| | After 6 | 11 minutes and | 15 minutes and | 15 minutes and | 24 minutes and |
| | months | 47 seconds | 13 seconds | 53 seconds | 20 seconds |

The results of the disintegration test in TABLE 1 have revealed that in EXAMPLE 1 of the invention, poly-γ-glutamic acid contained as the film component of the soft capsule inhibits the insolubilization phenomenon of the film that occurs during storage of the capsule.

### Test Example 2

### Calcium Solubilization Test

A calcium solubilization test was conducted by the following method using the capsules formed in EXAMPLE 1 and COMPARATIVE EXAMPLE 1. One capsule (Ca content 38 mg) was charged into 25 ml of purified water, and the solution was adjusted to pH of 1.5 with 1M hydrochloric acid. The resulting solution was warmed at 37° C. for 1 hour, then cooled to room temperature, and adjusted to pH of 8 with 1M sodium carbonate to obtain a sample solution with a volume adjusted to 50 ml. To 0.5 ml of this sample solution were added 0.25 ml of 20 mM disodium hydrogenphosphate and 0.75 ml of purified water, and the mixture was warmed at 37° C. for 1 hour. After the resulting mixture was cooled to room temperature, calcium phosphate formed was filtrated with a filter having a pore size of 0.45 μm, and the concentration of solubilized calcium in the filtrate was measured (Calcium C-Wako, manufactured by Wako Pure Chemical). Further, a calcium solubilization test was conducted using 95.5 mg of a calcium carbonate (Ca content 40%) powder instead of the capsule. The results are shown in TABLE 1. The solubilization ratio of calcium was calculated from the amount of calcium solubilized when the calcium content of one capsule was defined as 100%.

TABLE 2

| | | Comparative | |
|---|---|---|---|
| | Example 1 | Example 1 | Control |
| Sample | PGA-containing | Gelatin | Calcium carbonate |
| | film capsule | capsule | powder |
| Ca solubilization | 41% | 7% | 3% |
| ratio | | | |

The results of the calcium solubilization test in TABLE 2 have revealed that the capsule in EXAMPLE 1 of the invention shows the higher calcium solubilization ratio than the gelatin capsule in COMPARATIVE EXAMPLE 1 owing to poly-γ-glutamic acid contained as the film component. Accordingly, when the capsule in EXAMPLE 1 is administered, calcium taken in is absorbed in the body efficiently.

The invention is widely useful as an improved product of the existing gelatin capsule in the field in which capsules are used, for example, in the field of pharmaceutical preparations, foods and health foods. Further, the mineral ingredient-containing gelatin capsule excellent in easy mineral absorption enables mineral ingredients such as calcium to be effectively absorbed, and it is especially useful in the field of pharmaceutical preparations, foods and health foods for supplementing minerals in children at a growth age, preventing osteoporosis at an old age and the like.

1. A capsule film composition comprising gelatin and polyglutamic acid.

2. A gelatin capsule comprising the capsule film composition as claimed in claim 1.

3. The gelatin capsule as claimed in claim 2, wherein the polyglutamic acid is poly-γ-glutamic acid and/or poly-α-glutamic acid.

4. The gelatin capsule as claimed in claim 2 or 3, further comprising one or more capsule content ingredients selected from the group consisting of a protein, a vitamin, a mineral, a lipid, an herb, and a functional food material.

5. (canceled).

6. The gelatin capsule as claimed in claim 4, wherein said capsule content ingredient is a mineral.

7. The gelatin capsule as claimed in claim 6, wherein said mineral is calcium or iron.

8. The capsule film composition as claimed in claim 1, wherein the polyglutamic acid is poly-γ-glutamic acid and/or poly-α-glutamic acid.

9. The capsule film composition as claimed in claim 1, wherein the ratio of gelatin to polyglutamic acid is from 99:1 to 60:40, by weight.

10. The gelatin capsule as claimed in claim 2, wherein the ratio of gelatin to polyglutamic acid in the capsule film composition is from 99:1 to 60:40, by weight.

11. The capsule film composition as claimed in claim 1, wherein said capsule film composition further comprises one or more members selected from the group consisting of glycerin, a colorant and a screening agent.

12. The gelatin capsule as claimed in claim 2, wherein the capsule film composition further comprises one or more members selected from the group consisting of glycerin, a colorant and a screening agent.

13. A capsule film composition comprising gelatin and polyglutamic acid, wherein the ratio of gelatin to polyglutamic acid in the capsule film composition is from 99:1 to 60:40, by weight.

14. A gelatin capsule comprising a capsule film composition, wherein said capsule film composition comprises gelatin and polyglutamic acid in a ratio of from 99:1 to 60:40, by weight.

15. The capsule film composition as claimed in claim 13, wherein said capsule film composition further comprises one or more members selected from the group consisting of glycerin, a colorant and a screening agent.

16. The gelatin capsule as claimed in claim 14, wherein the capsule film composition further comprises one or more members selected from the group consisting of glycerin, a colorant and a screening agent.

17. The capsule film composition as claimed in claim 3, wherein the polyglutamic acid is poly-γ-glutamic acid and/or poly-α-glutamic acid.

18. The gelatin capsule as claimed in claim 14, wherein the polyglutamic acid comprising the capsule film composition is poly-γ-glutamic acid and/or poly-α-glutamic acid.
